(19) United States
(12) Patent Application Publication
(10) Pub. No.: US 2005/0215515 A1
(43) Pub. Date: Sep. 29, 2005

(54) Viscoelastic composition, method of use and package

(76) Inventors: Claudio Bucolo, Acicastello (IT); Melina G. Cro, Catania (IT); Adriana L. A. Maltese, Tremestie Etneo (IT)

Correspondence Address: Bausch & Lomb Incorporated, One Bausch & Lomb Place, Rochester, NY, 14604-2701, US

(21) Appl. No.: 10/812,544

(22) Filed: Mar. 29, 2004

(51) Int. Cl. A61K 31/728; A61K 31/737; A61K 31/716

(52) U.S. Cl. 514/54; 514/57

(57) ABSTRACT

The present invention is a viscoelastic composition, methods of use and a related device, wherein the viscoelastic composition comprises water, a minimum of about 0.01% w/v and a maximum of about 10% w/v of hyaluronic acid or a salt thereof and a minimum of about 0.01% w/v and a maximum of about 10% w/v of hydroxypropylmethylcellulose, wherein the viscoelastic comprises less than 0.01% w/v chondroitin sulfate.

### BACKGROUND OF THE INVENTION

1. Field of the Invention

The present invention relates to the field of viscoelastic compositions for use as ophthalmic viscosurgical devices.

2. Discussion of Related Art

In the past decade, advances in the technology of eye surgery have made surgical treatment of eye disease and deformities attractive to alternative therapies. One of the more common surgical procedures is cataract removal. Cataracts are opacities of the ocular lens, which generally arise in the elderly. Typically, cataract surgery involves removal of the cataractous lens from the capsular bag and replacement of the cataractous lens with a synthetic intraocular lens. Presently, this procedure involves making an incision through the sclera or cornea into the anterior chamber of the patient's eye. Another incision is made into the capsular bag. The cataractous lens is fractured in the capsular bag by procedures such as phacoemulsification and removed from the capsular bag by procedures such as aspiration. Thereafter, an intraocular lens is inserted into the capsular bag and deployed therein. The overall procedure is potentially traumatic to the tissue surrounding the anterior chamber. It is advantageous to reduce the amount of trauma to any living tissue in the patient's eye during a surgical procedure. Particularly, corneal endothelial cells in the capsular bag are sensitive to damage. Damage to the corneal endothelial cells is often permanent. Serious damage can cause loss of eyesight and failure of the surgical procedure.

Viscoelastic compositions are injected in the anterior chamber of the eye and the capsular bag during surgery to protect the tissue from physical trauma. The viscoelastic compositions provide a physical barrier or cushion between the instruments and the tissue. Furthermore, viscoelastic compositions maintain the space in a cavity during operation including the anterior chamber and capsular bag.

In addition to cataract surgery, viscoelastic compositions are useful in reducing tissue trauma and maintaining space in a cavity during other ophthalmic surgical procedures, including but not limited to trabeculectomy and vitrectomy.

Viscoelastic compositions have properties that make them effective for use in eye surgery to maintain the space in a cavity and to protect the tissue. A viscoelastic composition under zero-shear or low-shear preferably has a relatively high viscosity. Higher viscosity compounds under zero-shear or low-shear conditions have better space maintenance properties than low viscosity compounds (i.e. they maintain the space in a cavity into which they are injected). However, it is difficult to inject or remove a highly viscous liquid through a cannula used for surgical procedures inside the eye. It is highly desirable to have a compound that has low viscosity under high-shear conditions and high viscosity under zero-shear or low-shear conditions. Generally, the pseudoplasticity index is the ratio of the viscosity at zero-shear or low-shear condition to a higher-shear condition. It is desirable for a viscoelastic composition to have a high pseudoplasticity index.

Common viscoelastic compositions for eye surgery include sodium hyaluronate (Healong by Pfizer, New York, N.Y.), sodium hyaluronate and chondroitin sulfate (Viscoat® by Alcon Laboratories, Fort Worth, Tex.), hydroxypropylmethylcellulose (Ocucoat® by Bausch & Lomb, Rochester, N.Y.).

A composition whose viscoelastic component is essentially hydroxypropylmethylcellulose has excellent adherent properties and does not produce an inflammatory response. However, the space maintenance properties of hydroxypropylmethylcellulose could be improved upon.

A composition whose viscoelastic component is essentially sodium hyaluronate has good space maintaining characteristics. However, sodium hyaluronate in sufficient concentrations can induce an inflammatory response and cause IOP spikes after surgery.

A composition whose viscoelastic component is essentially sodium hyaluronate and chondroitin sulfate improves the adhesive properties over a sodium hyaluronate viscoelastic composition alone. However, the pseudoplasticity of the combination of sodium hyaluronate and chondroitin sulfate is lower than a sodium hyaluronate only viscoelastic composition.

U.S. Pat. Nos. 4,713,375, 5,013,714 and 5,204,325 disclose a viscoelastic composition for use in ophthalmic surgery such as cataract surgery. The viscoelastic composition comprises chondroitin sulfate and hydroxypropylmethylcellulose in different amounts. The viscous properties of hydroxypropylmethylcellulose, purportedly, are enhanced by the lubricious properties of chondroitin sulfate as set forth in this reference.

U.S. Pat. No. 5,366,964 discloses a viscoelastic composition for use in ophthalmic surgery that includes sodium hyaluronate, hydroxypropylmethylcellulose and chondroitin sulfate—each ingredient in amounts ranging from 0.01%-10%. A specific formulation was disclosed with 0.5% w/v chondroitin sulfate, 1.4% w/v hydroxypropylmethylcellulose and 0.05% w/v sodium hyaluronate.

European Patent Publication No. 0 516 901 discloses a serum-free medical solution for ex-vivo preservation of corneal tissue at low-temperatures. A glycosaminoglycan is combined with a deturgescent. Chondroitin sulfate and hyaluronic acid were two of several examples of a glycosaminoglycan. Hydroxypropylmethylcellulose was one of numerous examples of a deturgescent.

While significant improvements have been made in the Theological properties of viscoelastic compositions, there still exists a need for a composition that has good adhesive properties and more desirable Theological properties such as a high pseudoplasticity. Particularly, there exists a need for a viscoelastic composition that coats tissue effectively for protection while maintaining desirable physical properties for the injection, space maintenance and removal functions. The present invention addresses these and other needs.

### SUMMARY OF THE INVENTION

The present invention is a novel viscoelastic composition comprising water, hyaluronic acid or a salt thereof and hydroxypropylmethylcellulose. According to one embodiment, the viscoelastic composition has a minimum of about 0.01% w/v and a maximum of about 110% w/v of hyaluronic acid or a salt thereof and a minimum of about 0.01% w/v and a maximum of about 10% w/v of hydroxypropylmethylcellulose. The viscoelastic composition comprises less than 0.01% w/v chondroitin sulfate-preferably no chondroitin sulfate.

According to one embodiment of the present invention, there is a method of temporarily maintaining the space in a cavity in human tissue. The method comprises injecting a viscoelastic composition into the cavity. The viscoelastic composition comprises a minimum of about 0.01% w/v and a maximum of about 10% w/v hyaluronic acid or a salt thereof and a minimum of about 0.01% w/v and a maximum of about 10% w/v hydroxypropylmethylcellulose. The viscoelastic composition comprises less than 0.01% w/v chondroitin sulfate—preferably no chondroitin sulfate. Optionally or alternatively, a viscoelastic composition according to any embodiment, aspect, combination, concept or feature disclosed herein is injected into the cavity. Cavity space is maintained for a desired period of time. Thereafter, the viscoelastic composition is removed from the cavity.

In another embodiment of the present invention, there is a method of protecting tissue from trauma during a surgical procedure. The method includes coating at least a portion of the tissue with a viscoelastic composition comprising a minimum of about 0.01% w/v and a maximum of about 110% w/v hyaluronic acid or a salt thereof and a minimum of about 0.01% w/v and a maximum of about 10% w/v hydroxypropylmethylcellulose and less than less than 0.01% w/v chondroitin sulfate. Optionally or alternatively, a viscoelastic composition according to any embodiment, aspect, combination, concept or feature disclosed herein is used to coat the tissue. A surgical procedure is performed near the tissue after the tissue is coated. At least a portion of the viscoelastic composition is removed from the tissue after the surgical procedure is performed.

In one embodiment there is a method of replacing a natural lens from an eye. The method includes the step of providing a passage through a sclera into an anterior chamber of the eye. At least a portion of the aqueous humor is removed from the anterior chamber. A viscoelastic composition is injected into the anterior chamber. The viscoelastic composition comprises a minimum of about 0.01% w/v and a maximum of about 10% w/v hyaluronic acid or a salt thereof, a minimum of about 0.01% w/v and a maximum of about 10% w/v hydroxypropylmethylcellulose and less than 0.01% w/v chondroitin sulfate. Optionally or alternatively, a viscoelastic composition according to any embodiment, aspect, combination, concept or feature disclosed herein is injected, for example, into the anterior chamber of the eye. Then, the corneal lens is removed from the capsular bag of the eye. The viscoelastic composition is injected into the capsular bag. An intraocular lens is inserted into the capsular bag.

In still another embodiment, there is a package for a viscoelastic composition, the package comprises a syringe containing a viscoelastic composition comprising a minimum of about 0.01% w/v and a maximum of about 10% w/v hyaluronic acid or a salt thereof and a minimum of about 0.01% w/v and a maximum of about 10% w/v hydroxypropylmethylcellulose and less than 0.01% w/v chondroitin sulfate. Optionally or alternatively, a viscoelastic composition according to any embodiment, aspect, combination, concept or feature disclosed herein is contained in the syringe.

### BRIEF DESCRIPTION OF THE DRAWINGS

FIG. 1 is a graphic representation of the dynamic response of a viscoelastic composition according to the present invention (Formulation 1) compared to a viscoelastic composition known in the art (Formulation 6).

FIG. 2 is a graphic representation of the dynamic response of several compositions according to the present invention and one composition known in the art.

FIG. 3 is a graphic representation of the viscosity at various shear rates of a viscoelastic composition according to the present invention (Formulation 1) compared to a viscoelastic composition known in the art (Formulation 6).

### DETAILED DESCRIPTION OF THE INVENTION

Introduction

A viscoelastic composition, methods of use and a related device are the subject of the present invention. The viscoelastic composition comprises water, a minimum of about 0.01% w/v and a maximum of about 10% w/v of hyaluronic acid or a salt thereof and a minimum of about 0.01% w/v and a maximum of about 10% w/v of hydroxypropylmethylcellulose, wherein the viscoelastic composition comprises less than 0.01% w/v chondroitin sulfate. In one embodiment, the viscoelastic composition has a pseudoplasticity index having a minimum of about 60 and a maximum of about 9000.

Definitions

Hyaluronic acid is defined as a linear polysaccharide composed of alternating residues of the monosaccharides D-glucuronic acid and N-acetyl-D-glucosamine linked in repeating units. Salts of hyaluronic acid are defined as saccharides including monosaccharides, disaccharide, oligosaccharide and polysaccharides that includes hyaluronate salts including but not limited to sodium hyaluronate, potassium hyaluronate, calcium hyaluronate, magnesium hyaluronate.

Oligosaccharides are defined as saccharides that have 3 to 10 saccharide monomer units.

Polysaccharides are defined as saccharides that have 10 or more saccharide monomer units.

Pseudoplasticity index as used to define the subject matter of this application is defined as the ratio between viscosity at a 0.009 s⁻¹ shear rate and a viscosity at 369 s⁻¹ shear rate.

Zero-shear viscosity is defined as the extrapolation of the viscosity of a liquid to a zero-shear rate from measurements of viscosity as the shear rate approaches zero measured on a plate and cone rheometer at 34° C.

Medium-shear conditions are defined as shear conditions ranging from about 5 sec⁻¹ to about 50 sec⁻¹. Medium-shear viscosity, for the purpose this patent application, is defined as the viscosity of a liquid measured on a plate and cone rheometer at 34° C. with a shear rate of 10 sec⁻¹.

High-shear conditions are defined as shear conditions ranging from about 200 sec⁻¹ to about 1000 sec⁻¹. High-shear viscosity, for the purpose this patent application, is defined as the viscosity of a liquid measured on a plate and cone rheometer at 34° C. with a shear rate of 300 sec⁻¹.

A pseudoplastic material is defined as a material that has relatively high viscosity under low-shear and relatively low viscosity under high-shear conditions.

The phrase, “removing substantially all” as it relates to lenses and lens fragments is defined as removing a sufficient quantity to facilitate effective implantation of an intraocular lens. According to one embodiment, an effective removal of the lens requires a minimum of 90% w/v of the lens, 95% w/v of the lens or 98% w/v of the lens.

A cannula is defined as any tubular member having a passage that is configured to penetrate tissue and deliver a device through the passage.

A syringe is defined as a device having a reservoir, outlet and a piston received in the reservoir that is configured to be actuated by a force and compress the contents of the reservoir thereby expelling the contents of the reservoir through an outlet port, in one embodiment.

A chemical scavenger as used herein is a free radical scavengers and ion scavengers. A chemical scavenger reacts quickly with free radicals and other reactive ionic material and thereby prevents the free radicals and reactive ions from reacting with and/or damaging cellular tissue. Chemical scavengers that are preferable for use with a viscoelastic composition will not invoke a significant inflammatory response in the tissue to which it is injected. Furthermore, it is important that it does not interfere with the rheological properties of the viscoelastic composition.

The phrase, “viscosurgically pure,” as it refers to a viscoelastic composition or ingredient of a viscoelastic composition is defined as a level of purity that is sufficiently free of impurities to meet or exceed the United States Food and Drug Administration standards for a viscosurgical viscoelastic compositions at the time this application is filed.

Formulation

As noted, according to one embodiment of the present invention, there is a viscoelastic composition that comprises water, a minimum of about 0.01% w/v and a maximum of about 110% w/v of hyaluronic acid or a salt thereof and a minimum of about 0.01% w/v and a maximum of about 10% w/v of hydroxypropylmethylcellulose. The viscoelastic composition has less than 0.01% w/v chondroitin sulfate. Preferably, the viscoelastic composition has less than 0.005% w/v, less than 0.001% w/v or less than 0.0001% w/v chondroitin sulfate. Most preferably the viscoelastic composition has no chondroitin sulfate.

In one embodiment, the viscoelastic composition has a pseudoplasticity index that is a minimum of about 60 and a maximum of about 9000. Generally, the pseudoplasticity index is a minimum of about 80, about 100, about 120, about 160 and a maximum of about 5000, about 1000, about 500, about 400, about 300.

In another embodiment, the average molecular weight of the hyaluronic acid or a salt thereof in the viscoelastic composition is a minimum of about 500 kD and a maximum of about 5000 kD. Typically, the average molecular weight of the hyaluronic acid or a salt thereof is a minimum of about 600 kD, about 700 kD, about 800 kD or about 1000 kD. Typically, the average molecular weight of the hyaluronic acid or a salt thereof is a maximum of about 4000 kD, about 300 kD or about 2000 kD.

In still another embodiment, the average molecular weight of the hydroxypropylmethylcellulose in the viscoelastic composition is a minimum of about 10 kD and a maximum of about 120 kD. Typically, the average molecular weight of the hydroxypropylmethylcellulose is a minimum of about 11.5 kD, about 12 kD or about 20 kD. Typically, the average molecular weight of the hydroxypropylmethylcellulose is a maximum of about 90 kD, about 86 kD or about 60 kD.

The amount of hyaluronic acid or a salt thereof, of the present invention, is a minimum of about 0.1% w/v and a maximum of about 6% w/v based upon the total weight of the viscoelastic composition. Typically, the viscoelastic composition comprises a minimum amount of about 0.3% w/v, about 0.6% w/v, about 0.8% w/v or about 1.0% w/v hyaluronic acid or a salt thereof based upon the total weight of the viscoelastic composition. Typically, the viscoelastic composition comprises a maximum amount of about 5.0% w/v, about 4.0% w/v, about 3.0% w/v or about 2.0% w/v hyaluronic acid or a salt thereof based upon the total weight of the viscoelastic composition.

The amount of hydroxypropylmethylcellulose, according to one embodiment of the present invention, is a minimum of about 0.05% w/v and a maximum amount of about 5.0% w/v based upon the total weight of the viscoelastic composition. Typically, the viscoelastic composition has a minimum amount of about 0.1% w/v, about 0.3% w/v, about 0.4% w/v, about 0.6% w/v or about 0.8% w/v hydroxypropylmethylcellulose based upon the total weight of the viscoelastic composition. Typically, the viscoelastic composition has a maximum amount of about 4.0% w/v, about 3.0% w/v, about 2.0% w/v or about 1.0% w/v hydroxypropylmethylcellulose based upon the total weight of the viscoelastic composition.

The osmolality of the viscoelastic composition, in one embodiment, is a minimum of about 200 mOsmol/Kg and a maximum of about 400 mOsmol/Kg. Typically, the osmolality of the viscoelastic composition is a minimum of about 220 mOsmol/Kg, about 260 mOsmol/Kg, about 280 mOsmol/Kg, about 300 mOsmol/Kg or about 320 mOsmol/Kg. Typically, the osmolality of the viscoelastic composition is a maximum of about 400 mOsmol/Kg, about 380 mOsmol/Kg, about 360 mOsmol/Kg or about 340 mOsmol/Kg.

The zero-shear viscosity of one embodiment of the viscoelastic composition is a minimum of about 6×10⁴ cps and a maximum of about 4×10⁶ cps. Generally, the zero-shear viscosity of the viscoelastic composition is a minimum of about 1×10⁵ cps, about 4×10⁵ cps or about 8×10⁵ cps. Generally, the zero-shear viscosity of the viscoelastic composition is a maximum of about 3.5×10⁶ cps, about 2.5×10⁶ cps, about 1.8×10⁶ CpS, about 1.2×10⁶ cps or about 9×10⁵ CpS.

The medium-shear viscosity of another embodiment of the viscoelastic composition is a minimum of about 10000 cps and a maximum of about 30000 cps. Generally, the medium-shear viscosity of the viscoelastic composition is a minimum of about 11000 cps, about 12000 cps or about 13000 cps. Generally, the medium-shear viscosity of the viscoelastic composition is a maximum of about 25000 cps, about 23000 cps, about 20000 cps or about 18000 cps.

The high-shear viscosity of the viscoelastic composition is a minimum of about 500 cps and a maximum of about 2000 cps. Generally, the high-shear viscosity of the viscoelastic composition is a minimum of about 550 cps, about 600 cps or about 700 cps. Generally, the high-shear viscosity of the viscoelastic composition is a maximum of about 1500 cps, about 1300 cps, about 1100 cps or about 1000 cps.

The viscoelastic composition according to one concept of the invention has a ratio of hydroxypropylmethylcellulose to hyaluronic acid or a salt thereof in a range having a minimum of about 0.01 and a maximum of about 20. Typically, the ratio of hydroxypropylmethylcellulose to hyaluronic acid or a salt thereof in the viscoelastic composition is in a range having a minimum of about 0.03, about 0.1, about 0.3 or about 0.5. Typically, the ratio of hydroxypropylmethylcellulose to hyaluronic acid or a salt thereof in the viscoelastic composition is in a range having a maximum of about 10, about 5, about 2 or about 1.

The viscoelastic composition of another concept of the present invention further comprises a chemical scavenger. Chemical scavengers include but are not limited to tris[hydroxymethyl]aminomethane, polyols, glutatione, ascorbate, vitamin E, BHA, BHT, propylgallate, β-carotene, trolox, metabisulfite, flavonoids, sodium formate, thiourea, carbohydrates, 2-mercaptoethanol, dimethylsulfoxide, imidazole, dimethylthiourea, SOD, salicylate, proline, indoles, sulforaphane, polyphenols, citrate, cysteine and derivatives thereof. Preferable chemical scavengers include but are not limited to sorbitol, tris[hydroxymethyl]aminomethane, mannitol, hexahydric alcohol. Most preferable chemical scavengers include sorbitol and tris[hydroxymethyl]aminomethane. In one embodiment, the viscoelastic composition comprises a minimum sorbitol concentration of about 0.1% w/v, about 0.3% w/v, about 0.5% w/v or about 1% w/v and a maximum sorbitol concentration of about 10% w/v, about 6% w/v, about 4% w/v or about 3% w/v. The minimum tris[hydroxymethyl] aminomethane concentration is about 0.1 mM, about 0.3 mM, about 0.5 mM or about 1 mM and the maximum tris[hydroxymethyl] aminomethane concentration is 40 mM, about 30 mM, about 20 mM or about 15 mM.

The pH of the viscoelastic composition is a minimum of about 5 and a maximum of about 8. In one embodiment, the pH of the viscoelastic composition is a minimum of about 5, about 5.5, about 6 or about 6.5 and a maximum of about 8, about 7.5, about 7.2 or about 7.

The viscoelastic composition of one embodiment has a formulation set forth in Table 1.

TABLE 1

| Component of Property of | |
|---|---|
| Composition | Amount |
| 1.0 × 10⁶-3 × 10⁶ Molecular Weight | 0.5% w/v to 3% w/v |
| Hyaluronic Acid or Salt Form Thereof | |
| 20,000-200,000 Molecular Weight | 0.1% w/v to 2% w/v |
| Hydroxypropylmethylcellulose | |
| Sorbitol | 0.1% w/v to 20% w/v |
| Tris[hydroxymethyl]aminomethane | 1 mM to 100 mM |
| Buffered to pH | 6.9 to 7.5 |
| Osmolality adjusted to | 290-350 mOsm/Kg |

In one preferred embodiment the viscoelastic composition comprises the following:
2.3% w/v hyaluronic acid (MW 1.98×10⁶)
0.8% w/v hydroxypropylmethylcellulose (MW 86,000)
4.4% w/v sorbitol
20 mM tris[hydroxymethyl]aminomethane
purified water q. s. to 100 ml
pH 7.3
335 mOsm/Kg
   In another preferred embodiment, the viscoelastic composition comprises the following:
2% w/v hyaluronic acid (MW 1.98×10⁶)
0.8% w/v hydroxypropylmethylcellulose (MW 86,000)
4.4% w/v sorbitol
20 mM tris[hydroxymethyl] aminomethane
purified water q. s. to 100 ml
pH 7.3
335 mOsm/Kg
   In another preferred embodiment, the viscoelastic composition comprises the following:

2% w/v hyaluronic acid (MW 1.98×10⁶)
1% w/v hydroxypropylmethylcellulose (MW 86,000)
4.4% w/v sorbitol
20 mM tris[hydroxymethyl] aminomethane
purified water q. s. to 100 ml
pH 7.3
335 mOsm/Kg

Methods of Use

Viscoelastic composition according to any one or more of the foregoing embodiments, concepts or aspects including combinations and variations of the foregoing embodiments, aspects, concepts, combinations and features of the present invention can be used with one or more of the following methods.

According to one embodiment of the present invention, there is a method of temporarily maintaining the space in a cavity in human tissue. The method comprises injecting a viscoelastic composition into the cavity. The viscoelastic composition comprises a minimum of about 0.01% w/v and a maximum of about 10% w/v hyaluronic acid or a salt thereof and a minimum of about 0.01% w/v and a maximum of about 10% w/v hydroxypropylmethylcellulose. The viscoelastic composition comprises less than 0.01% w/v chondroitin sulfate-preferably no chondroitin sulfate. Optionally or alternatively, a viscoelastic composition according to any embodiment, aspect, combination, concept or feature disclosed herein is injected into the cavity. Cavity space is maintained for a desired period of time. Thereafter, the viscoelastic composition is removed from the cavity. In one embodiment, the cavity is the anterior chamber of the eye or the capsular bag.

In another embodiment of the present invention, there is a method of protecting tissue from trauma during a surgical procedure. The method includes coating at least a portion of the tissue with a viscoelastic composition comprising a minimum of about 0.01% w/v and a maximum of about 10% w/v hyaluronic acid or a salt thereof and a minimum of about 0.01% w/v and a maximum of about 10% w/v hydroxypropylmethylcellulose and less than less than 0.01% w/v chondroitin sulfate. Optionally or alternatively, a viscoelastic composition according to any embodiment, aspect, combination, concept or feature disclosed herein is used to coat the tissue. A surgical procedure is performed near the tissue after the tissue is coated. At least a portion of the viscoelastic composition is removed from the tissue after the surgical procedure is performed. In another embodiment the step of coating the tissue covers at least a portion of the tissue in a capsular bag of an eye.

In one embodiment, there is a method of replacing a natural lens from an eye. Examples of procedures for removing a lens from a patient's eye include but are not limited to U.S. Pat. No. 3,589,363 (cataract surgery), U.S. Pat. No. 3,693,613 (phacoemulsification) and U.S. Pat. No. 5,718,676 (process using micro flow needle), which are all incorporated herein by reference in their entirety. The process generally includes providing a passage through a sclera or cornea into an anterior chamber of the eye. The process involves making a small incision into the sclera or cornea. Alternatively or additionally, a cannula or trochar is used to create a passage through the sclera or cornea. Preferably, the incision or passage is as small as possible. Preferably the incision or passage is smaller than about 5 mm, about 4 mm or about 3 mm. Thereafter, the aqueous humor is withdrawn or otherwise removed from the anterior chamber of the eye.

A viscoelastic composition is injected into the anterior chamber. The viscoelastic composition comprises a minimum of about 0.01% w/v and a maximum of about 10% w/v hyaluronic acid or a salt thereof, a minimum of about 0.01% w/v and a maximum of about 10% w/v hydroxypropylmethylcellulose and less than 0.01% w/v chondroitin sulfate. Optionally or alternatively, a viscoelastic composition according to any embodiment, aspect, combination, concept or feature disclosed herein is injected, for example, into the anterior chamber of the eye. The viscoelastic composition, of one embodiment, maintains the space in the anterior chamber. The viscoelastic composition of another embodiment, coats the tissue in the wall of the anterior chamber.

According to one embodiment, there is a package for a viscoelastic composition that includes a delivery device. The device delivers a viscoelastic composition into the anterior chamber of a patient's eye. The device includes a syringe that contains a viscoelastic composition comprising a minimum of about 0.01% w/v and a maximum of about 10% w/v hyaluronic acid or a salt thereof, a minimum of about 0.01% w/v and a maximum of about 10% w/v hydroxypropylmethylcellulose and less than 0.01% w/v chondroitin sulfate. Optionally or alternatively, the syringe contains a viscoelastic composition according to any embodiment, aspect, combination, concept or feature disclosed herein.

The syringe further comprises an outlet port and, optionally, a cannula configured to sealably connect to the outlet port. The cannula has a maximum inner diameter of about 2 mm. Typically, the maximum inner diameter is about 1.8 mm, about 1.5 mm or about 1 mm. Generally, the minimum inner diameter is about 0.8 mm, about 0.6 mm or about 0.4 mm.

In one embodiment, the viscoelastic composition requires a maximum force of 30 N to pass through a stainless steel cannula having a length of 2.2 cm and an inner diameter of 0.5 mm at a delivery rate of 0.02 ml/sec. Preferably, the viscoelastic composition requires a maximum force of about 27 N, about 25 N, about 20 N or about 18 N to pass through a stainless steel cannula having a length of 2.2 cm and an inner diameter of 0.5 mm at a delivery rate of 0.02 ml/sec.

Once the viscoelastic composition is inserted into the anterior chamber the corneal lens is removed. The technique for removing the lens includes performing a capsulorhexis incision and breaking down the lens into smaller pieces through phacoemulsification or other known techniques. Thereafter, the pieces are removed by aspiration.

The viscoelastic composition is inserted into the capsular bag for space maintenance purposes. Moreover, the viscoelastic composition coats the capsular bag and protects it for additional steps in the surgical procedure.

According to one embodiment, the intraocular lens is inserted into the capsular bag. Typically, there is a method of inserting an intraocular lens into a capsular bag of an eye. The method comprises providing a lens insertion device comprising a loadable chamber configured to receive the intraocular lens, a tapered conduit having a first end connected to the loadable chamber and a second end. The second end is configured to penetrate through the passage in the corneal lens and into the capsular bag. An example of a lens insertion device is found in U.S. Pat. No. 6,558,419, which is incorporated herein by reference in its entirety. The lens insertion device is further configured with a slidable actuator. The slidable actuator of one embodiment is configured to actuate the intraocular lens through the conduit past the second end. Typically, the second end of the tapered conduit has an inner diameter that is a maximum of about 5 mm. Preferably the second end of the tapered conduit has an inner diameter that is a maximum of about 4 mm about 3.5 mm, about 3 mm or about 2.8 mm.

Prior to deployment, at least a portion of the intraocular lens is coated with a viscoelastic composition according to any one of the embodiments of the present invention. The intraocular lens is loaded into the loadable chamber either before or after it is coated. The conduit is inserted through the passage. The actuator forces the intraocular lens through the passage and into the capsular bag. After the intraocular lens is deployed, the conduit is removed from the passage.

Typically, at least a portion of the viscoelastic composition is removed from the capsular bag and/or anterior chamber. A physiological solution is then used to fill the anterior chamber. The sclera and/or cornea are sutured to close the passage.

### EXAMPLES

### Example 1

The following formulation was prepared and labeled Formulation 1
2.3% w/v hyaluronic acid (MW 1.98×10⁶)
0.8% w/v hydroxypropylmethylcellulose (MW 86,000)
4.4% w/v sorbitol
20 mM tris[hydroxymethyl]aminomethane (“tris”)
Purified water q. s. to 100 ml
pH 7.3
335 mOsm/Kg

### Example 2

The following formulation was prepared and labeled Formulation 2
2.3% w/v hyaluronic acid (MW 1.98×10⁶)
0.8% w/v hydroxypropylmethylcellulose (MW 86,000)
Purified water q. s. to 100 ml
<pH 7.3
<300 mOsm/Kg

### Example 3

The following formulation was prepared and labeled Formulation 3
2.3% w/v hyaluronic acid (MW 1.98×10⁶)
1% w/v hydroxypropylmethylcellulose (MW 86,000)
20 mM tris
Purified water q. s. to 100 ml
pH 7.3
<335 mOsm/Kg

### Example 4

The following formulation was prepared and labeled Formulation 4
2.3% w/v hyaluronic acid (MW 1.98×10⁶)
0.8% w/v hydroxypropylmethylcellulose (MW 86,000)
4.4% w/v sorbitol
Purified water q. s. to 100 ml
<pH 7.3
<335 mOsm/Kg

### Example 5

The following formulation was prepared and labeled Formulation 5:
2.3% w/v hyaluronic acid (MW 1.98×10⁶)
4.4% w/v sorbitol
20 mM tris[hydroxymethyl]aminomethane (“tris”)
Purified water q. s. to 100 ml
<pH 7.3
<335 mOsm/Kg

### Example 6

A commercial sample of Viscoat® was identified as Formulation 6.

### Example 7

### Rheological Measurements

The small-amplitude oscillatory shear measurements were performed to evaluate the dynamic response of each of the formulations disclosed in Example 1 through 6 to determine their linear viscoelastic properties. The rheological properties were evaluated on a Bohlin VOR Rheometer at controlled temperature of 34° C. The geometry was cone and plate and the plate was forced to oscillate at increasing frequency.

In the dynamic experiment, the mechanical response, expressed as shear stress of each sample is intermediate between an ideal pure elastic solid and ideal pure viscous fluid and is described by two moduli G′ and G″ as function of oscillation frequency. G′ is the shear storage modulus (or elastic modulus) and gives information about the elasticity or the energy stored during deformation. G″ is the shear loss modulus (or viscous modulus) and describes the viscous character or the energy dissipated as heat. A viscoelastic composition has a predominantly viscous character (G″>G′) at low frequencies and essentially elastic behavior (G′>G″) at higher frequencies. This transition, indicated by the crossing of the G′ and G″ curves, occurs at a given value of the frequency is referred to as the crossover frequency.

TABLE 2
Cross-over Frequency

| | G′ | G″ | Cross-over frequency |
|---|---|---|---|
| | (Pa) | (Pa) | (Hz) |
| Formulation 1 | 85 | 81 | 0.04 |
| Formulation 6 | 170 | 150 | 3 |

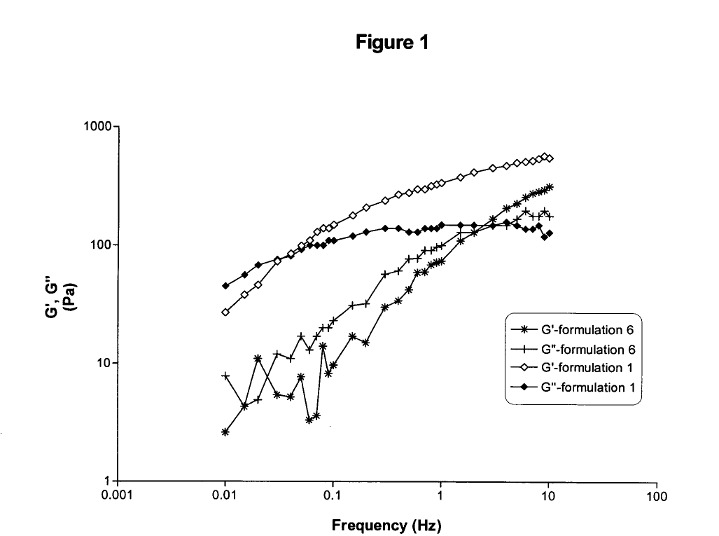

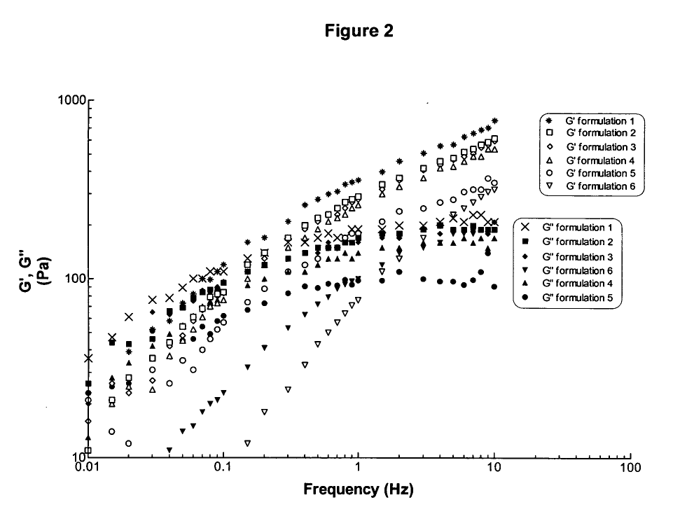

FIG. 1 shows the dynamic response of Formulation 1 and Formulation 6. Formulation 1 has both moduli higher than Viscoat and the crossing of the moduli curves occurring at lower frequency as shown in Table 2. This means that G′>G″ for a wider range of frequencies and hence Formulation 1 is more elastic than Formulation 6. FIG. 2 shows the dynamic response of Formulations 1 through 6.

The results showed that dynamic response increase in this order: Formulation 1 hade the highest dynamic response increase followed in descending order by Formulation 2, Formulation 3, Formulation 4, Formulation 5 and Formulation 6. Thus, all of the variations of hyaluronic acid and hydroxypropylmethylcellulose have a higher dynamic response increase than hyaluronic acid without hydroxypropylmethylcellulose and the commercial formula for Viscoat®.

### Example 8

### Steady Shear Measurements

The non-linear flow properties of the investigated materials were evaluated through steady shear measurements to determine the viscosity η as function of shear rate. Steady shear properties of Formulation 1 and Formulation 6 were evaluated on a Bohlin VOR Rheometer at controlled temperature of 34° C. The geometry was cone and plate and the plate was forced to rotate at increasing shear rate. In the steady shear experiment, the pseudoplastic behavior of Formulation 1 and Formulation 6 was evaluated.

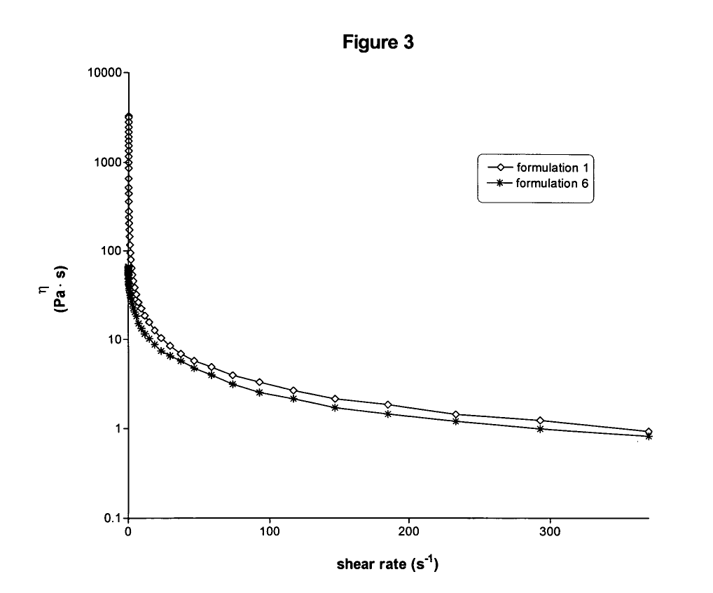

Pseudoplasticity describes the characteristic of a viscoelastic composition when, under the application of shear force, is converted from a gel-like, highly viscous state to a low viscous, watery-state. The pseudoplasticity index (i.e. the ratio between viscosity at 0.009 s⁻¹ and viscosity at 369 s⁻¹ shear rate) is a measure of the pseudoplastic behavior. FIG. 3 shows that Formulation 1 has a higher viscosity at all shear rate than Formulation 6. Table 3 illustrates that Formulation 1 with a pseudoplasticity index of 3551 is higher than Formulation 6 with a pseudoplasticity index of 77.

TABLE 3
Pseudoplasticity Index

| | Viscosity (mPa s) | Viscosity (mPa s) | Pseudoplasticity |
|---|---|---|---|
| | at 0.009 s⁻¹ | at 369 s⁻¹ | index |
| Formulation 1 | 3310000 | 932 | 3551 |
| Formulation 6 | 63700 | 825 | 77 |

### Example 9

### Humor Aqueous Replacement

Biomicroscopic effects on the anterior segment in the rabbit eye after humor aqueous substitution with Formulation 1 were evaluated. A quantity of 0.2 ml of Formulation 1 was exchanged simultaneously with an equal volume of aqueous fluid in the right eye of six (6) subject rabbits. The left eye received buffered salt solution (BSS) as a control. Biomicroscopic evaluation was performed by a slit-lamp (Sbisà, mod. 4179) every day for seven days. Treated eyes remained unchanged in all aspects, and no differences were observed when compared with control eyes.

Although preferred embodiments have been depicted and described in detail, it will be apparent to those skilled in the relevant art that the specification including the examples are made without the intention of limiting the scope of the invention and that various modifications, additions, substitutions, and the like can be made without departing from the spirit of the invention and these are therefore considered to be within the scope of the invention as defined in the claims which follow.

1. A viscoelastic composition comprising water, a minimum of about 0.01% w/v and a maximum of about 10% w/v of hyaluronic acid or a salt thereof and a minimum of about 0.01% w/v and a maximum of about 10% w/v of hydroxypropylmethylcellulose, wherein the viscolelastic comprises less than 0.01% w/v chondroitin sulfate and has a pseudoplasticity index having a minimum of about 60 and a maximum of about 9000.

2. The composition of claim 1, wherein the average molecular weight of the hyaluronic acid or a salt thereof is a minimum of about 500 kD and a maximum of about 5000 kD.

3. The composition of claim 1, wherein the average molecular weight of the hydroxypropylmethylcellulose is a minimum of about 10 kD and a maximum of about 120 kD.

4. The composition of claim 1, wherein the viscoelastic composition comprises a minimum amount of about 0.1% w/v and a maximum amount of about 6% w/v, hyaluronic acid or a salt thereof based upon the total weight of the viscoelastic composition.

5. The composition of claim 1, wherein the viscoelastic composition has a minimum amount of about 0.05% w/v and a maximum amount of about 5.0% w/v hydroxypropylmethylcellulose, based upon the total weight of the viscoelastic composition.

6. The composition of claim 1, wherein the osmolality of the viscoelastic composition is a minimum of about 200 mOsmol/Kg and a maximum of about 400 mOsmol/Kg.

7. The composition of claim 1, wherein the zero-shear viscosity of the viscoelastic composition is a minimum of about 6×10⁴ cps and a maximum of about 4×10⁶ cps.

8. The composition of claim 1, wherein the medium-shear viscosity of the viscoelastic composition is a minimum of about 10000 cps and a maximum of about 30000 cps.

9. The composition of claim 1, wherein the high-shear viscosity of the viscoelastic composition is a minimum of about 500 cps and a maximum of about 2000 cps.

10. The composition of claim 1, wherein the viscoelastic composition has a ratio of hydroxypropylmethylcellulose to hyaluronic acid or a salt thereof in a range having a minimum of about 0.01 and a maximum of about 20.

11. The composition of claim 1, wherein the viscoelastic composition further comprises a chemical scavenger. Chemical scavengers include but are not limited to tris[hydroxymethyl] aminomethane, polyols, glutatione, ascorbate, vitamin E, BHA, BHT, propyl gallate, β-carotene, trolox, metabisulfite, flavonoids, sodium formate, thiourea, carbohydrates, 2-mercaptoethanol, dimethylsulfoxide, imidazole, dimethylthiourea, SOD, salicylate, proline, indoles, sulforaphane, polyphenols, citrate, cysteine and derivatives thereof.

12. The composition of claim 1, wherein the pH of the viscoelastic composition is a minimum of about 5 and a maximum of about 8.

13. A method of temporarily maintaining the space in a cavity in human tissue, the method comprising the steps of:
(a) injecting a viscoelastic composition into the cavity, the viscoelastic composition comprises a minimum of about 0.01% w/v and a maximum of about 10% w/v hyaluronic acid or a salt thereof and a minimum of about 0.01% w/v and a maximum of about 10% w/v hydroxypropylmethylcellulose, wherein the viscoelastic composition comprises less than 0.01% w/v chondroitin sulfate; and
(b) removing the viscoelastic composition from the cavity.

14. The method of claim 13, wherein the average molecular weight of the hyaluronic acid or a salt thereof is a minimum of about 1000 kD and a maximum of about 3000 kD.

15. The method of claim 13, wherein the average molecular weight of the hydroxypropylmethylcellulose is a minimum of about 12 kD and a maximum of about 86 kD.

16. The method of claim 13, wherein the viscoelastic composition comprises a minimum amount of about 1% w/v and a maximum amount of about 3% w/v, hyaluronic acid or a salt thereof based upon the total weight of the viscoelasfic composition.

17. The method of claim 13, wherein the viscoelastic composition has a minimum amount of about 0.1% w/v and a maximum amount of about 2% w/v hydroxypropylmethylcellulose, based upon the total weight of the viscolelastic material.

18. The method of claim 13, wherein the osmolality of the viscoelastic composition is a minimum of about 200 mOsmol/Kg and a maximum of about 400 mOsmol/Kg.

19. The method of claim 13, wherein the zero-shear viscosity of the viscoleastic material is a minimum of about 8×10⁵ cps and a maximum of about 3.5×10⁶ cps.

20. The method of claim 13, wherein the medium-shear viscosity of the viscoelastic composition is a minimum of about 13000 cps and a maximum of about 25000 cps.

21. The method of claim 13, wherein the high-shear viscosity of the viscoelastic composition is a minimum of about 700 cps and a maximum of about 1300 cps.

22. The method of claim 13, wherein the viscoelastic composition has a ratio of hydroxypropylmethylcellulose to hyaluronic acid or a salt thereof in a range having a minimum of about 0.1 and a maximum of about 20.

23. The method of claim 13, wherein the viscoelastic composition further comprises a chemical scavenger.

24. The method of claim 13, wherein the pH of the viscoelastic composition is a minimum of about 6.5 and a maximum of about 7.5.

25. The method of claim 13, wherein the cavity is the anterior chamber of the eye or the capsular bag.

26. A method of protecting tissue from trauma during a surgical procedure, the method comprising the steps of:
(a) coating at least a portion of the tissue with a viscoelastic composition comprising a minimum of about 0.01% w/v and a maximum of about 10% w/v hyaluronic acid or a salt thereof and a minimum of about 0.01% w/v and a maximum of about 10% w/v hydroxypropylmethylcellulose, wherein the viscoelastic composition comprises less than 0.01% w/v chondroitin sulfate;
(b) performing a surgical procedure near the tissue after the step of (a) coating; and
(c) removing at least a portion of the viscoelastic composition from the tissue after the step (b) performing.

27. The method of claim 26, wherein the step of (a) coating covers at least a portion of the tissue in an anterior chamber of an eye.

28. The method of claim 26, wherein the step of (a) coating covers at least a portion of the tissue in a capsular bag of an eye.

29. The method of claim 26, wherein the average molecular weight of the hyaluronic acid or a salt thereof is a minimum of about 1000 kD and a maximum of about 3000 kD.

30. The method of claim 26, wherein the average molecular weight of the hydroxypropylmethylcellulose is a minimum of about 12 kD and a maximum of about 86 kD.

31. The method of claim 26, wherein the viscoelastic composition comprises a minimum amount of about 1% w/v and a maximum amount of about 3% w/v, hyaluronic acid or a salt thereof based upon the total weight of the viscoelastic composition.

32. The method of claim 26, wherein the viscoelastic composition has a minimum amount of about 0.1% w/v and a maximum amount of about 2% w/v hydroxypropylmethylcellulose, based upon the total weight of the viscolelastic material.

33. The method of claim 26, wherein the osmolality of the viscoelastic composition is a minimum of about 200 mOsmol/Kg and a maximum of about 400 mOsmol/Kg.

34. The method of claim 26, wherein the zero-shear viscosity of the viscoleastic material is a minimum of about 8×10⁵ cps and a maximum of about 3.5×10⁶ cps.

35. The method of claim 26, wherein the medium-shear viscosity of the viscoelastic composition is a minimum of about 13000 cps and a maximum of about 25000 cps.

36. The method of claim 26, wherein the high-shear viscosity of the viscoelastic composition is a minimum of about 700 cps and a maximum of about 1300 cps.

37. The method of claim 26, wherein the viscoelastic composition has a ratio of hydroxypropylmethylcellulose to hyaluronic acid or a salt thereof in a range having a minimum of about 0.1 and a maximum of about 20.

38. The method of claim 26, wherein the viscoelastic composition further comprises a chemical scavenger.

39. The method of claim 26, wherein the pH of the viscoelastic composition is a minimum of about 6.5 and a maximum of about 7.5.

40. A package for a viscoelastic composition, the package comprising a syringe containing a viscoelastic composition comprising a minimum of about 0.01% w/v and a maximum of about 10% w/v hyaluronic acid or a salt thereof and a minimum of about 0.01% w/v and a maximum of about 10% w/v hydroxypropylmethylcellulose, wherein the viscoelastic composition comprises less than 0.01% w/v chondroitin sulfate.

41. The package of claim 40, wherein the syringe has an outlet port, the package further comprising a cannula configured to sealably connect to the outlet port having a maximum inner diameter of about 2 mm. Typically, the maximum inner diameter is about 1.8 mm, about 1.5 mm or about 1 mm. Generally, the minimum inner diameter is about 0.8 mm, about 0.6 mm or about 0.4 mm.

42. The package of claim 40, wherein viscoelastic composition requires a maximum force of 30 N to pass through a stainless steel cannula having a length of 2.2 cm and an inner diameter of 0.5 mm at a delivery rate of 0.02 ml/sec. Preferably, the viscoelastic composition requires a maximum force of about 27 N, about 25 N, about 20 N or about 18 N to pass through a stainless steel cannula having a length of 2.2 cm and an inner diameter of 0.5 mm at a delivery rate of 0.02 ml/sec.

43. The package of claim 40, wherein the average molecular weight of the hyaluronic acid or a salt thereof is a minimum of about 1000 kD and a maximum of about 3000 kD.

44. The package of claim 40, wherein the average molecular weight of the hydroxypropylmethylcellulose is a minimum of about 12 kD and a maximum of about 86 kD.

45. The package of claim 40, wherein the viscoelastic composition comprises a minimum amount of about 1% w/v and a maximum amount of about 3% w/v, hyaluronic acid or a salt thereof based upon the total weight of the viscoelastic composition.

46. The package of claim 40, wherein the viscoelastic composition has a minimum amount of about 0.1% w/v and a maximum amount of about 2% w/v hydroxypropylmethylcellulose, based upon the total weight of the viscolelastic material.

47. The package of claim 40, wherein the osmolality of the viscoelastic composition is a minimum of about 200 mOsmol/Kg and a maximum of about 400 mOsmol/Kg.

48. The package of claim 40, wherein the zero-shear viscosity of the viscoleastic material is a minimum of about 8×10⁵ cps and a maximum of about 3.5×10⁶ cps.

49. The package of claim 40, wherein the medium-shear viscosity of the viscoelastic composition is a minimum of about 13000 and a maximum of about 25000.

50. The package of claim 40, wherein the high-shear viscosity of the viscoelastic composition is a minimum of about 700 and a maximum of about 1300.

51. The package of claim 40, wherein the viscoelastic composition has a ratio of hydroxypropylmethylcellulose to hyaluronic acid or a salt thereof in a range having a minimum of about 0.1 and a maximum of about 20.

52. The package of claim 40, wherein the viscoelastic composition further comprises a chemical scavenger.

53. The package of claim 40, wherein the pH of the viscoelastic composition is a minimum of about 6.5 and a maximum of about 7.5.

54. A method of replacing a natural lens from an eye, the method comprising the steps of:
(a) providing a passage through a sclera into an anterior chamber of the eye;
(b) removing at least a portion of the aqueous humor from the anterior chamber;
(c) inserting a viscoelastic composition into the anterior chamber, the viscoelastic composition comprises a minimum of about 0.01% w/v and a maximum of about 10% w/v hyaluronic acid or a salt thereof and a minimum of about 0.01% w/v and a maximum of about 10% w/v hydroxypropylmethylcellulose, wherein the viscoelastic composition comprises less than 0.01% w/v chondroitin sulfate;
(d) removing the corneal lens from the capsular bag of the eye;
(e) injecting the viscoelastic composition into the capsular bag; and
(f) inserting an intraocular lens into the capsular bag.

55. The method of claim 54, further comprising the step of removing at least a portion of the viscoelastic composition from the capsular bag.

56. The method of claim 54, further comprising the step of removing at least a portion of the viscoelastic composition from the anterior chamber.

57. The method of claim 54, further comprising the step of suturing the sclera after the step (g) inserting an intraocular lens.

58. The method of claim 54, wherein the average molecular weight of the hyaluronic acid or a salt thereof is a minimum of about 1000 kD and a maximum of about 3000 kD.

59. The method of claim 54, wherein the average molecular weight of the hydroxypropylmethylcellulose is a minimum of about 12 kD and a maximum of about 86 kD.

60. The method of claim 54, wherein the viscoelastic composition comprises a minimum amount of about 1% w/v and a maximum amount of about 3% w/v, hyaluronic acid or a salt thereof based upon the total weight of the viscoelastic composition.

61. The method of claim 54, wherein the viscoelastic composition has a minimum amount of about 0.1% w/v and a maximum amount of about 2% w/v hydroxypropylmethylcellulose, based upon the total weight of the viscolelastic material.

62. The method of claim 54, wherein the osmolality of the viscoelastic composition is a minimum of about 200 mOsmol/Kg and a maximum of about 400 mOsmol/Kg.

63. The method of claim 54, wherein the zero-shear viscosity of the viscoleastic material is a minimum of about 8×10⁵ cps and a maximum of about 3.5×10⁶ cps.

64. The method of claim 54, wherein the medium-shear viscosity of the viscoelastic composition is a minimum of about 13000 and a maximum of about 25000.

65. The method of claim 54, wherein the high-shear viscosity of the viscoelastic composition is a minimum of about 700 and a maximum of about 1300.

66. The method of claim 54, wherein the viscoelastic composition has a ratio of hydroxypropylmethylcellulose to hyaluronic acid or a salt thereof in a range having a minimum of about 0.1 and a maximum of about 20.

67. The method of claim 54, wherein the viscoelastic composition further comprises a chemical scavenger.

68. The method of claim 54, wherein the pH of the viscoelastic composition is a minimum of about 6.5 and a maximum of about 7.5.
